(19) United States
(12) Patent Application Publication
(10) Pub. No.: US 2025/0064464 A1
(43) Pub. Date: Feb. 27, 2025

(54) FLUID CONTROL DEVICES FOR CLOT TREATMENT SYSTEMS, AND ASSOCIATED SYSTEMS AND METHODS

(71) Applicant: Inari Medical, Inc., Irvine, CA (US)

(72) Inventors: Mikayla Ann Barkley, Newport Beach, CA (US); Schuyler Rideout, San Clemente, CA (US); Benjamin Edward Merritt, San Clemente, CA (US)

(21) Appl. No.: 18/812,966

(22) Filed: Aug. 22, 2024

Related U.S. Application Data
(60) Provisional application No. 63/578,235, filed on Aug. 23, 2023.

(51) Int. Cl. A61B 17/22; A61B 17/00

(52) U.S. Cl. CPC A61B 17/22; A61B 2017/00292; A61B 2017/00477; A61B 2017/22079; A61B 2217/005

(57) ABSTRACT

Disclosed herein are fluid control devices for clot treatment systems, and associated systems and methods. In some embodiments, a fluid control device can be coupled between a catheter and a pressure source and configured to selectively allow or prevent a vacuum generated within the pressure source to be applied to a lumen of the catheter. The catheter and the fluid control device can together define a lumen or fluid path to the pressure source. The lumen or fluid path can have an at least generally uniform dimension (e.g., diameter) along its length, which is expected to inhibit or even prevent choke points or other resistances to fluid flow through the fluid control device, including when used with large bore catheters.

### CROSS-REFERENCE TO RELATED APPLICATION

The present application claims priority to U.S. Provisional Patent Application No. 63/578,235, filed Aug. 23, 2023, and titled “FLUID CONTROL DEVICES FOR CLOT TREATMENT SYSTEMS, AND ASSOCIATED SYSTEMS AND METHODS,” which is incorporated by reference herein in its entirety.

### TECHNICAL FIELD

The present technology generally relates to fluid control devices for use in systems for treating occlusive (e.g., clot) material within a human patient, and associated systems and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries-a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Various systems exist for performing a thrombectomy or removing occlusive material to reestablish blood flow within a patient. Some of these systems use (i) a pressure source to generate and store a vacuum and (i) a fluid control device, such as a stopcock, to apply the vacuum to a catheter to aspirate the clot material. Traditionally, the fluid control device is connected to a side port or proximal hub that is much smaller than the catheter's lumen, even when attached to larger bore catheters (greater than 20Fr in diameter). However, such small fluid control devices create “choke points” in the fluid path through the system and, in turn, can reduce performance during aspiration. Some systems use large bore stopcocks to attempt to solve these problems. However, as the bore size increases, these stopcocks become exponentially more challenging to design and build while maintaining reasonable ergonomics. Moreover, the increased surface area of the stopcock valve element needed to accommodate these larger bore sizes, and the seals formed therewith, creates much more friction when trying to turn the stopcock handle. Additionally, relative to small bore stopcocks, large bore stopcocks need to be under more compression to effectively seal high vacuum pressures; these components relax over time in response to such compressive forces, thereby reducing their effectiveness and shelf life.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system configured in accordance with embodiments of the present technology.

FIGS. 2A-2F are different views of a fluid control device configured in accordance with embodiments of the present technology.

FIG. 3 is a side view of a tubing assembly including the fluid control device of FIGS. 2A-2F in accordance with embodiments of the present technology.

FIGS. 4A-4E are different views of a fluid control device configured in accordance with additional embodiments of the present technology.

FIGS. 5A-5C are different views of a fluid control device configured in accordance with additional embodiments of the present technology.

FIGS. 6A-6C are different views of a fluid control device configured in accordance with additional embodiments of the present technology.

FIGS. 7A and 7B are different views of another fluid control device configured in accordance with embodiments of the present technology.

### DETAILED DESCRIPTION

The present technology is generally directed to fluid control devices for clot treatment systems, and associated systems and methods. In some embodiments, a clot treatment system includes a catheter, a fluid control device, and a pressure source. The catheter can be fluidly coupled to one side of the fluid control device and the pressure source can be fluidly coupled to another side of the fluid control device. The catheter and the fluid control device can together define a lumen or fluid path to the pressure source. The fluid control device can be moved (e.g., actuated by a user) between (i) a first configuration in which the fluid control device prevents a vacuum generated within the pressure source from being applied to the catheter and (ii) a second configuration in which the fluid control device allows the vacuum to be applied to the catheter, e.g., to aspirate clot material from within a patient. The lumen or fluid path can have an at least generally uniform dimension (e.g., diameter) along its length, which is expected to inhibit or even prevent choke points or other resistances to fluid flow through the fluid control device, including when used with large bore catheters.

The fluid control devices of the present technology include features that are expected to improve their operation and/or otherwise make them easier to use during an aspiration-based clot removal procedure. For example, at least some of the fluid control devices include a sloped or angled valve component configured to engage a sloped or angled surface within the fluid control device, e.g., to form a substantially fluid-impermeable seal. This interaction between angled surfaces is expected to improve the quality of the seal. As another example, at least some of the fluid control devices include a valve component (e.g., a spherical valve component) positioned within a body of the fluid control device but spaced apart from one or more inner surfaces of the body. This is expected to reduce a resistance to rotating the valve component relative to the body, e.g., to transition the fluid control device between the first and second configurations.

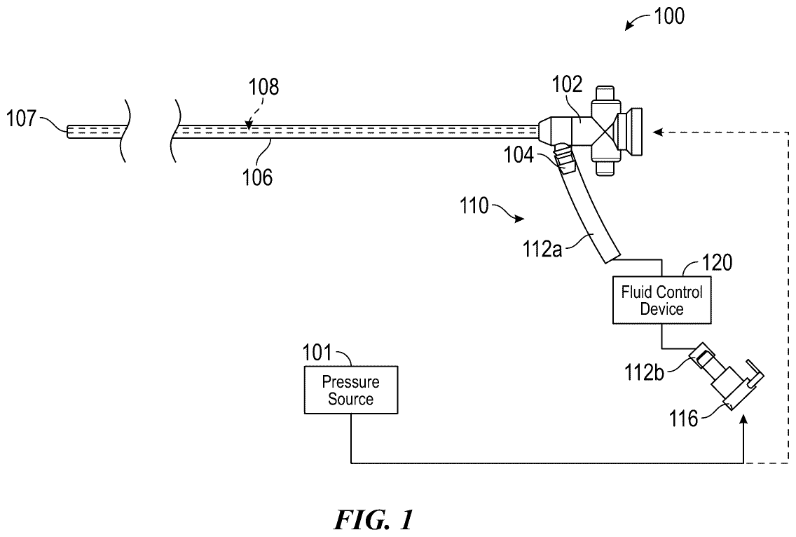

Certain details are set forth in the following description and in FIGS. 1-7B to provide a thorough understanding of various embodiments of the present technology. In other instances, well-known structures, materials, operations, and/or systems often associated with intravascular procedures, clot removal procedures, clot treatment systems, clot treatment devices, fluid control devices, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, tubing assembly 110 is first introduced and discussed with reference to FIG. 1.

To the extent any materials incorporated herein by reference conflict with the present disclosure, the present disclosure controls.

FIG. 1 is a partially schematic side view of a clot treatment system 100 (“the system 100”) configured in accordance with embodiments of the present technology. The system 100 can also be referred to as an aspiration assembly, a vascular access system, a clot removal system, a thrombectomy system, and/or the like. In the illustrated embodiment, the system 100 includes a tubing assembly 110 fluidly coupled to a catheter 106 via a valve 102. In some embodiments, the catheter 106 is an elongate member (e.g., a sheath, a shaft) configured to be inserted into and through a patient's vasculature and used to, for example, remove or otherwise treat clot material therein. In other embodiments, the catheter 106 can be an introducer sheath configured to be inserted through the skin and tissue tract of the patient to provide an access site through which other components (e.g., other catheters used to treat clot material) can traverse to easily access the vasculature. Accordingly, while referred to as “catheter 106,” the catheter 106 can comprise an introducer sheath, an access sheath, and/or another type of elongate member configured to be inserted through the skin and tissue tract and/or to traverse the vasculature of a patient. In these and/or other embodiments, the catheter 106 can be a large bore catheter, e.g., having an outer diameter equal to or greater than 16 Fr, such as up to 20 Fr, 22 Fr, 24 Fr, 26 Fr, 28 Fr, 30 Fr, or 32 Fr. In general, the system 100 (i) can include features generally similar in structure and/or function, or identical in structure and/or function, to those of the clot treatment systems described in detail in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

The catheter 106 further defines a lumen 108 (shown in dashed line in FIG. 1) extending entirely therethrough, e.g., from the valve 102 to a distal terminus 107 of the catheter 106. The catheter 106 can have varying lengths, flexibilities, shapes, thicknesses, and/or other properties along its length. For example, the catheter 106 can comprise one or more coils, braids, and/or other structures positioned between one or more liner layers (e.g., inner and outer liner layers). In some embodiments, the catheter 106 can include several features generally similar or identical in structure and/or function to any of the catheters described in U.S. patent application Ser. No. 17/529,018, titled “CATHETERS HAVING SHAPED DISTAL PORTIONS, AND ASSOCIATED SYSTEMS AND METHODS,” and filed Nov. 17, 2021, U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, and/or U.S. patent application Ser. No. 18/159,507, titled “ASPIRATION CATHETERS HAVING GROOVED INNER SURFACE, AND ASSOCIATE SYSTEM AND METHODS,” and filed Jan. 25, 2023, each of which is incorporated by reference herein in its entirety.

The valve 102 is fluidly coupled to the lumen 108 of the catheter 106 and can be integral with or coupled to the catheter 106 such that these components move together. In some embodiments, the valve 102 is a hemostasis valve that is configured to maintain hemostasis during a clot treatment procedure by preventing fluid flow in a proximal direction through the valve 102 as various components such as dilators, delivery sheaths, pull members, guidewires, interventional devices, other aspiration catheters, and so on are inserted through the valve 102 to be delivered through the catheter 106 to a treatment site in a blood vessel. The valve 102 can include a branch or side port 104 configured to fluidly couple the lumen 108 of the catheter 106 to the tubing assembly 110. In some embodiments, the valve 102 can be a valve of the type disclosed in U.S. patent application Ser. No. 16/536,185, now U.S. Pat. No. 11,000,682, filed Aug. 30, 2018, and titled “HEMOSTASIS VALVES AND METHODS OF USE,” which is incorporated herein by reference in its entirety.

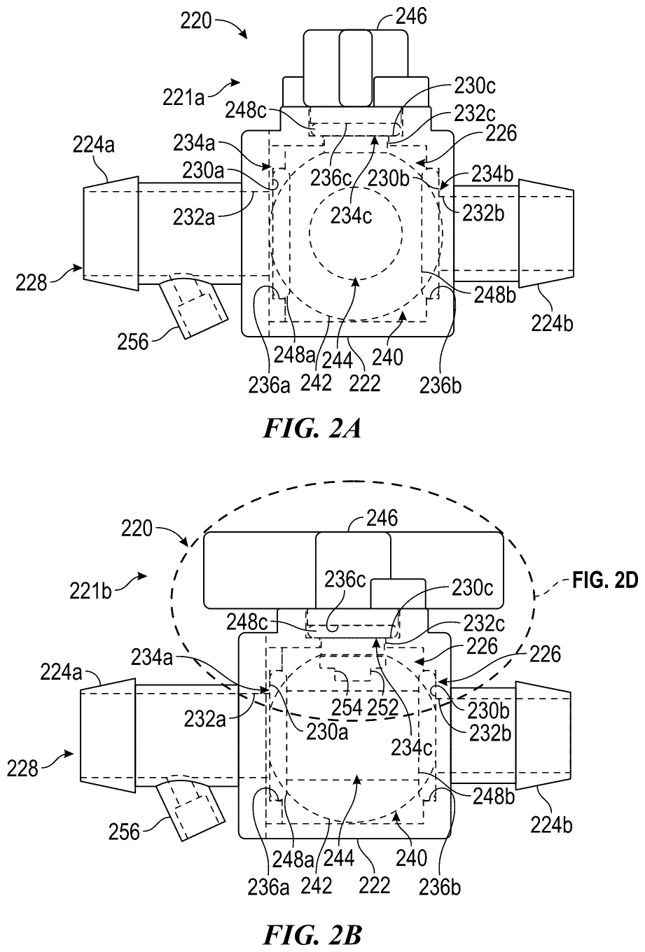

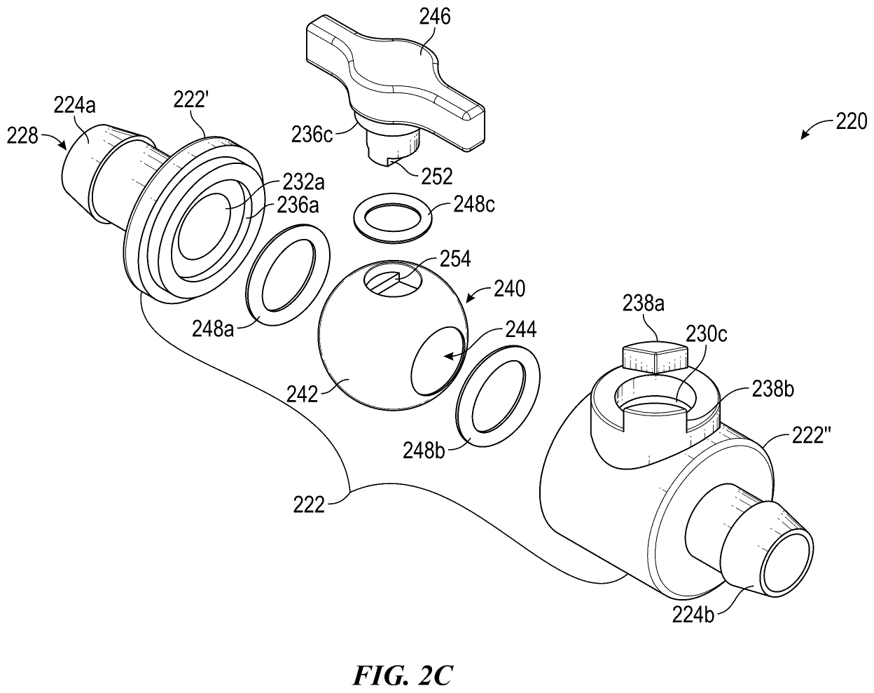

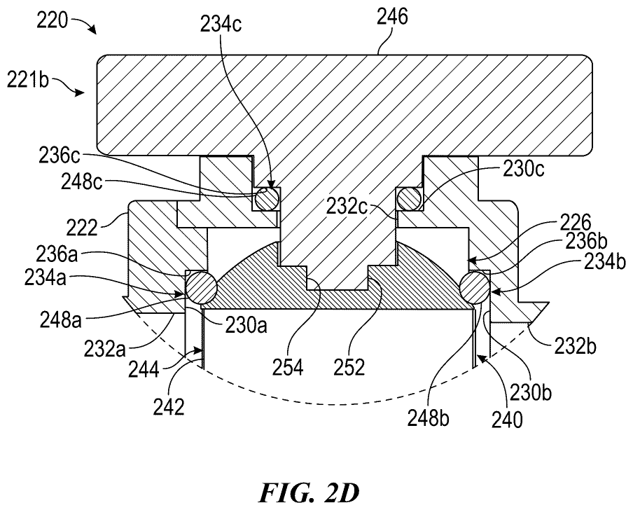

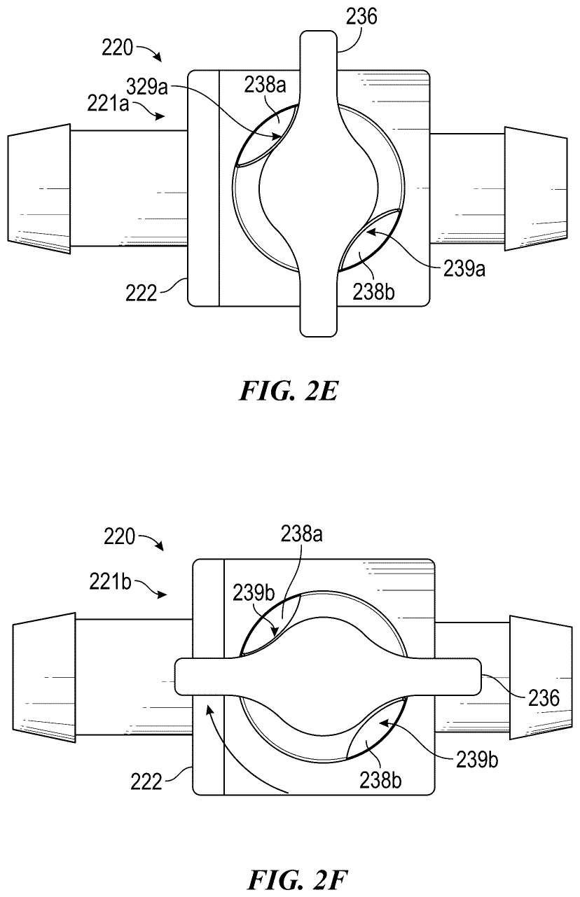

In the illustrated embodiment, the tubing assembly 110 fluidly couples the catheter 106 to a pressure source 101, such as a syringe. In at least some embodiments, the tubing assembly 110 and the catheter 106 have a same or substantially same inner dimension, e.g., to define a lumen or flow path of uniform or substantially uniform diameter extending from the distal terminus 107 of the catheter to the pressure source 101. The pressure source 101 can be configured to generate (e.g., form, create, charge, build-up) a vacuum (e.g., negative relative pressure) and store the vacuum for subsequent application to the catheter 106. The tubing assembly 110 can include one or more tubing sections 112 (individually labeled as a first tubing section 112a and a second tubing section 112b), at least one fluid control device or valve 120, and at least one connector 116 (e.g., a Toomey tip connector) for fluidly coupling the tubing assembly 110 to the pressure source 101 and/or other suitable components. In some embodiments, the connector 116 is a quick-release connector (e.g., a quick disconnect fitting) that enables rapid coupling/decoupling of the catheter 106 and the fluid control device 120 to/from the pressure source 101. In some embodiments, the fluid control device 120 is fluidly coupled to (i) the side port 104 of the valve 102 via the first tubing section 112a and (ii) the connector 116 via the second tubing section 112b. The fluid control device 120 is externally operable by a user to regulate the flow of fluid therethrough and, specifically, from the lumen 108 of the catheter 106 to the pressure source 101. For example, the fluid control device 120 can be transitioned between (i) a first or closed configuration in which the fluid control device 120 inhibits or even prevents fluid flow therethrough and (ii) a second or open configuration in which fluid can flow through the fluid control device 120. These and/or other aspects of the fluid control devices of the present technology are described in greater detail below with reference to FIGS. 2A-7B.

During a clot removal procedure, at least a portion of the system 100, including at least a portion of the catheter 106, can be inserted through the vasculature of a patient. In some embodiments, the system 100 is inserted, through an introducer sheath that traverses the skin and tissue of the patient to provide an access site, to a target treatment location proximate to the clot material. The fluid control device 120 can be in the closed position during insertion of the catheter 106. After positioning the catheter 106 at the treatment location and with the fluid control device 120 in the closed position, a user/operator can generate a vacuum in the pressure source 101 by, for example, withdrawing the plunger of a syringe coupled to the connector 116. In this manner, a vacuum is charged within the pressure source 101 (e.g., a negative pressure is maintained) before the pressure source 101 is fluidly connected to the lumen 108 of the catheter 106. To aspirate the lumen 108 of the catheter 106, the user can actuate (e.g., open) the fluid control device 120 to fluidly connect the pressure source 101 to the catheter 106 and thereby apply or release the vacuum stored in the pressure source 101 to the lumen 108 of the catheter 106. Opening of the fluid control device 120 instantaneously or nearly instantaneously applies the stored vacuum pressure to the tubing assembly 110 and the catheter 106, thereby generating a suction pulse throughout the catheter 106 that can aspirate the clot material into the catheter 106. In some embodiments, the vacuum from the pressure source 101 is applied with the fluid control device 120 in an open position (e.g., to provide continuous vacuum). That is, the user can generate the vacuum in the pressure source 101 while the fluid control device 120 is open (e.g., while the pressure source 101 is fluidly connected to the lumen 108 of the catheter 106) to thereby aspirate the clot material while also simultaneously generating the vacuum, e.g., without or substantially without storing the vacuum in the pressure source 101.

FIGS. 2A-2F are different views of a fluid control device 220 configured in accordance with embodiments of the present technology. More specifically, FIG. 2A is a side view of the fluid control device 220 in a closed configuration 221a. FIG. 2B is a side view of the fluid control device 220 in an open configuration 221b. FIG. 2C is a perspective exploded view of the fluid control device 220 in the open configuration 221b. FIG. 2D is a side cross-sectional view of a region 2D shown in FIG. 2B. FIG. 2E is a top view of the fluid control device 220 in the closed configuration 221a. FIG. 2F is a top view of the fluid control device 220 in the open configuration 221b. At least some aspects of the fluid control device 220 can be at least generally similar or identical in structure and/or function to the fluid control device 120 of FIG. 1. The fluid control device 220 can be used in the system 100 if FIG. 1 in the same or similar manner as the fluid control device 120.

Referring to FIGS. 2A-2D together, the fluid control device 220 includes a body 222 and a gating or fluid control assembly 240. The body 222 is shown as partially transparent in FIGS. 2A and 2B for clarity. The body 222 can include a first or distal connector portion 224a and a second or proximal connector portion 224b opposite (e.g., directly opposite) the first connector portion 224a. The body 222 defines a chamber 226 at least partially between the first connector portion 224a and the second connector portion 224b, and a lumen 228 extending through the body 222, e.g., between and/or fully through the first connector portion 224a and the second connector portion 224b. The chamber 226 can be cylindrical with a rectangular prism or cubical cross-sectional shape in at least some embodiments, or another suitable shape in other embodiments. The lumen 228 can include a first or distal opening 232a (e.g., to the chamber 226) formed through a first or distal surface 230a within the body 222 and second or proximal opening 232b (e.g., to the chamber 226) formed through a second or proximal surface 230b within the body 222. In at least some embodiments, the lumen 228 has a same dimension as the lumen 108 of the catheter 106 (FIG. 1).

The gating assembly 240 can be positioned within, or at least partially within, the chamber 226 of the body 222, e.g., at least partially between the first surface 230a and the second surface 230b. The gating assembly 240 includes a valve component 242 that defines a lumen 244. The valve component 242 can have a spherical shape in at least some embodiments, or another suitable shape in other embodiments. The valve component 242 can be configured to rotate relative to the body 222 of the fluid control device 220 to transition the fluid control device 220 between the open configuration 221b (FIGS. 2B and 2C) and the closed configuration 221a (FIG. 2A). When the fluid control device 220 is in the open configuration 221b (FIGS. 2B and 2C), the lumen 244 of the fluid control device body 222 can be aligned (e.g., colinear, coaxial) with the lumen 228 of the body 222, and/or one or both of the first opening 232a and the second opening 232b, to allow fluid flow through the gating assembly 240 and/or otherwise between the first connector portion 224a and the second connector portion 224b. When the fluid control device 220 is in the closed configuration 221a (FIG. 2A), the gating assembly lumen 244 can be rotated out of alignment with the lumen 228 of the body 222 to inhibit or even prevent fluid flow through the gating assembly 240 and/or otherwise between the first connector portion 224a and the second connector portion 224b. In FIG. 2B, for example, the gating assembly lumen 244 is rotated 90° relative to the lumen 228 when the fluid control device 220 is in the closed configuration 221a.

In some embodiments, the gating assembly 240 includes a handle 246 coupled to the valve component 242 that can be grasped by a user to rotate the valve component 242 to transition the fluid control device 220 between the open configuration 221b and the closed configuration 221a. Referring to FIGS. 2B-2D, the handle 246 can include a tab 252 (e.g., a protrusion, projection) and the valve component 242 can include a corresponding recess 254 configured (e.g., shaped, sized) to receive the tab 252 therein to rotationally secure the handle 246 to the valve component 242. The tab 252 can be secured within the recess 254 via adhesives, fasteners, welding, press-fitting, and/or the like such that rotation of the handle 246 rotates the valve component 242 to transition the fluid control device 220 between the open configuration 221b and the closed configuration 221a. At least a portion of the handle 246 (e.g., the tab 252) can extend through a third or upper opening 232c formed through a third or upper surface 230c of the body 222 to couple to the valve component 242.

Referring to FIGS. 2A, 2B and 2D, one or more sealing elements 248a-c (e.g., O-rings) can be positioned between and/or in contact with at least a portion of the gating assembly 240 and the body 222. Individual ones of the sealing elements 248a-c can form a substantially fluid-impermeable seal between at least the portion of the gating assembly 240 and the body 222. In the illustrated embodiment, for example, a first sealing element 248a is positioned between the valve component 242 and the first surface 230a to form a first seal 234a therebetween, a second sealing element 248b is positioned between the valve component 242 and the second surface 230b to form a second seal 234b therebetween, and a third sealing element 248c is positioned between the handle 246 and the third surface 230c to form a third seal 234c therebetween. The first sealing element 248a can be held in contact with (e.g., compressed against) the valve component 242 by a first annular retaining wall 236a extending from (e.g., perpendicularly from) the first surface 230a. Similarly, the second sealing element 248b can be held in contact with (e.g., compressed against) the valve component 242 by a second annular retaining wall 236b extending from (e.g., perpendicularly from) the second surface 230b. Accordingly, positioning the valve component 242 within the chamber can press the first and second sealing elements 248a, 248b against the first and second surfaces 230a, 230b to form the first and second seals 234a, 234b, respectively. The third sealing element 248c can be held in contact with the third surface 230c by a third annular retaining wall 236c of the handle 246. In some embodiments, coupling the handle 246 to the valve component 242 can cause the third annular retaining wall 236c to press the third sealing element 248c against the third surface 230c to form the third seal 234c.

When the fluid control device 220 is in the closed configuration 221a or the open configuration 221b, one or both of the first seal 234a and the second seal 234b can inhibit or even prevent fluid within the lumen 228 from entering the chamber 226, e.g., so that all or substantially all fluid that enters the fluid control device 220 via the first connector portion 224a flows through the gating assembly lumen 244 and/or exits the fluid control device 220 via the second connector portion 224b. In some instances, however, fluid (e.g., residual fluid, liquid residue, etc.) within the first connector portion 224a, the second connector portion 224b, or the gating assembly lumen 244 may enter the chamber 226, e.g., when the fluid control device 220 is transitioned between the closed configuration 221a and the open configuration 221b. In these and/or other instances, the third seal 234c can be a backup seal that inhibits or even prevents any such fluid that enters the chamber 226 from leaking out of the body 222, e.g., through the third opening 232c.

The various components of the fluid control device 220 can comprise plastic, metal, and/or other suitably rigid materials. In the illustrated embodiment, the valve component 242 has a spherical shape. In some aspects of the present technology, the spherical shape can at least partially reduce, or eliminate entirely, the surface area of the valve component 242 that is in contact with one or more inner surfaces of the body 222 defining the chamber 226. For example, the sealing elements 248a-c can support the valve component 242 within the chamber 226 so that all, or at least a portion, of the surface area of the valve component 242 is spaced apart from the body 222 (e.g., not contacting the surfaces of the body 222 that define the chamber 226). Accordingly, there is no plastic-to-plastic or other interface between the body 222 and the valve component 242 as in some conventional fluid control devices that increases friction and resistance to rotation. Moreover, because the valve component 242 forms the seals 234 (e.g., via compression) with the sealing elements 248a, material creep and/or other deformation due to plastic-to-plastic contact is expected to be reduced or eliminated entirely. In some embodiments, the sealing elements 248a-c can be covered in a lubricant, such as silicone grease, to further reduce resistance to rotation.

In some aspects of the present technology, reducing the resistance of the gating assembly 240 to rotation can be particularly beneficial when the fluid control device 220 defines a large lumen 228, 244 for use with a large bore catheter (e.g., the catheter 106 of FIG. 1). As described herein, increasing the size of the lumen of the catheter 106 can improve clot removal and ingestion. However, to maintain the diameter of the lumen through the system 100, the lumens 228, 244 through the fluid control device 220 must have a corresponding large diameter and thus require a relatively larger fluid control device 220. Friction within conventional fluid control devices that, for example, include contact between a rotational valve component and body of the fluid control device, increases with increasing component sizes such that it can be difficult for a user to open/close the fluid control device.

Referring to FIGS. 2A and 2B, in some embodiments the fluid control device 220 includes a side port 256 fluidly coupled to the lumen 228 and configured to allow a user to insert (e.g., inject) fluid into at least a portion of the fluid control device 220. In the illustrated embodiment, the first connector portion 224a includes the side port 256 so that the user can insert fluid distally of the gating assembly 240. In other embodiments, the second connector portion 224b and/or another suitable portion of the fluid control device 220 can include the side port 256.

As best illustrated in FIG. 2C, the body 222 of the fluid control device can include a first or proximal body portion 222′ and a second or distal body portion 222″. The first body portion 222′ and the second body portion 222″ can be coupled together using glue, ultrasonic welding, or another suitable technique.

Referring to FIGS. 2C, 2E, and 2F together, in some embodiments the body 222 includes one or more alignment or stop features 238a-b positioned to contact the handle 246 to confine rotation of the handle 246 between the closed configuration 221a and the open configuration 221b. For example, in the closed configuration 221a shown in FIG. 2E, the alignment features 238a-b can contact first side portions 239a of the handle 236 to inhibit or even prevent further rotation of the handle 236. This can indicate to a user that the fluid control device 220 is in the closed configuration 221a. Likewise, in the open configuration 221b shown in FIG. 2F, the alignment features 238a-b can contact second side portions 239b of the handle 236 to inhibit or even prevent further rotation of the handle 236. This can indicate to the user that the fluid control device 220 is in the open configuration 221b.

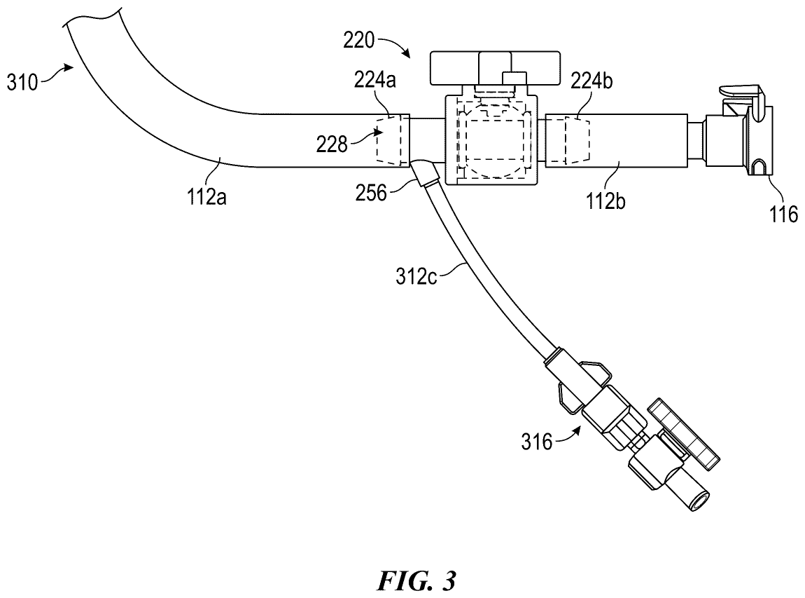

FIG. 3 is a side view of a tubing assembly 310 including the fluid control device 220 in accordance with embodiments of the present technology. The tubing assembly 310 can include at least some features that are at least generally similar or identical in structure and/or function to the tubing assembly 110 of FIG. 1. For example, the tubing assembly 310 includes the first tubing section 112a, the second tubing section 112b, and the connector 116. The first tubing section 112a can be coupled to the first connector portion 224a of the fluid control device 220. The second tubing section 112b can connect the second connector portion 224b of the fluid control device 220 to the connector 116. In at least some embodiments, the lumen 228 of the fluid control device 220 has a same, or an at least generally similar, diameter as an inner diameter of the tubing assembly 110. Accordingly, the fluid control device 220 and the tubing assembly 310 can together define a lumen or flow path of at least generally uniform diameter extending to the pressure source 101 (FIG. 1). This, in turn, is expected to inhibit or even prevent the formation of “choke points” or other resistances to fluid flow through the tubing assembly 310 and the fluid control device 220, thereby improving clot ingestion and aspiration.

The tubing assembly 310 can further include a third tubing section 312c fluidly coupled between the side port 256 and a second connector 316. In the illustrated embodiment the second connector 316 includes a quick connector configured to be connected to a source of fluid, to receive one or more other syringes, couple to one or more other tubing sections, etc. Accordingly, in at least some embodiments, the user can couple the fluid source to the second connector 316 to introduce fluid into the tubing assembly 310 and/or the system 100 (FIG. 1) to, for example, flush the tubing assembly 310 and/or the system 100, e.g., after aspirating clot material from within a patient. Additionally, or alternatively, the user can use the side port 256 to perform a number of other actions, including introducing contrast fluid, flushing or de-airing the system 100 (FIG. 1) and/or reinjecting blood (e.g., filtered blood) into the patient. Although in the illustrated embodiment the side port 256 is configured to receive the third tubing section 312c at least partially within the side port 256, in other embodiments the side port 256 can be barbed, flared, and/or otherwise configured to receive the third tubing section 312c over and/or at least partially around the side port 256.

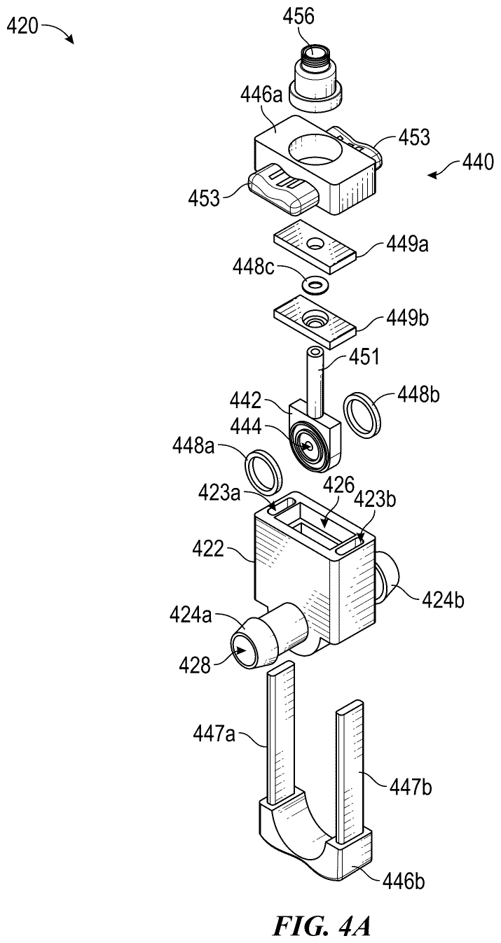

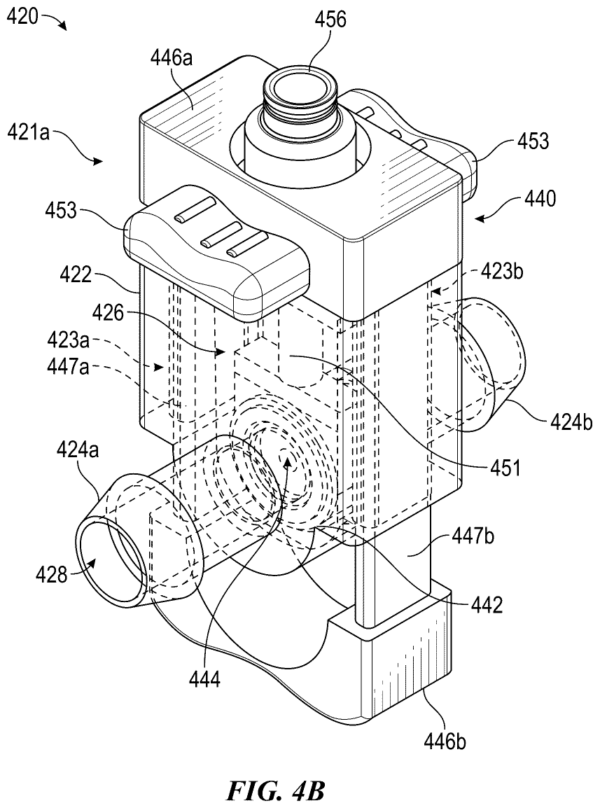

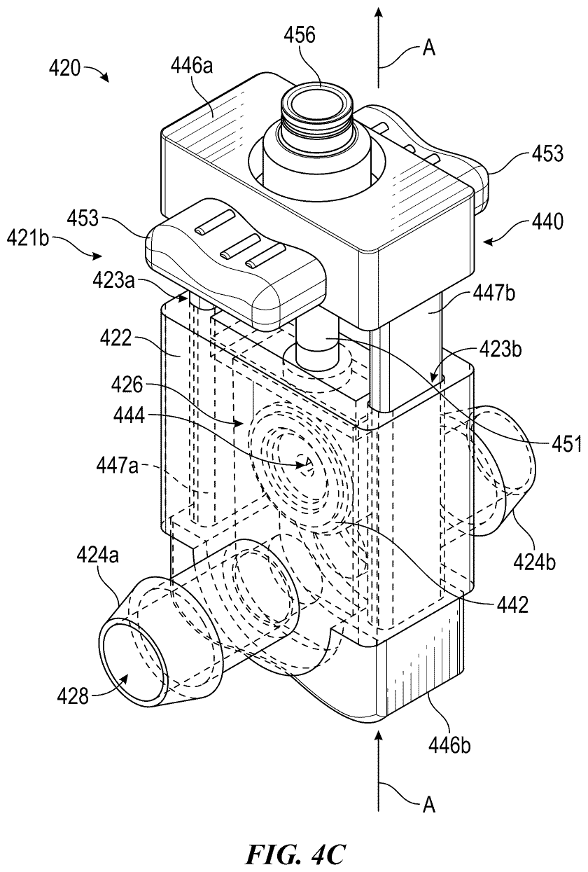

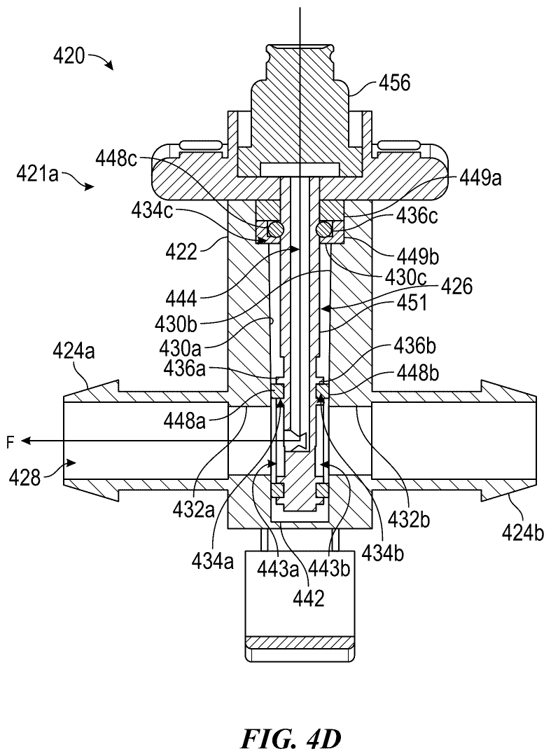

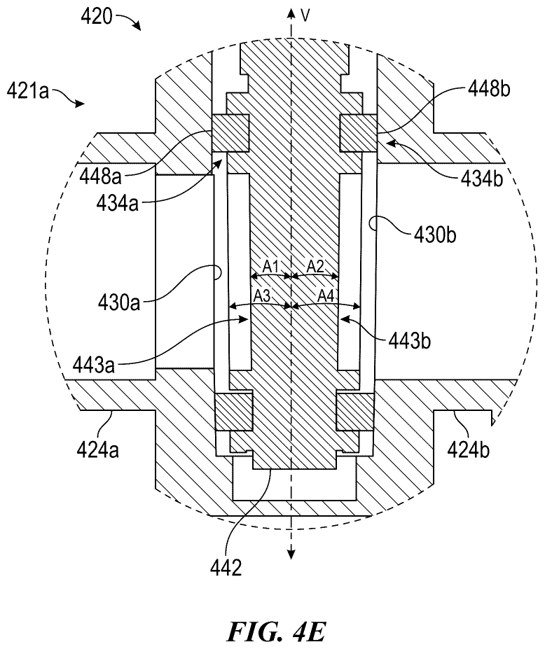

FIGS. 4A-4E are different views of a fluid control device 420 configured in accordance with additional embodiments of the present technology. FIG. 4A is a perspective exploded view of the fluid control device 420. FIG. 4B is a perspective view of the fluid control device 420 in a closed configuration 421a. FIG. 4C is a perspective view of the fluid control device 420 in an open configuration 421b. FIGS. 4D and 4E are enlarged side cross-sectional views of the fluid control device 420. At least some aspects of the fluid control device 420 can be at least generally similar or identical in structure and/or function to the fluid control device 120 of FIG. 1 and/or the fluid control device 220 of FIGS. 2A-3. For example, referring to FIGS. 4A-4C together, the fluid control device 420 includes a body 422 and a gating or fluid control assembly 440. The body 422 is shown as partially transparent in FIGS. 4A and 4B for clarity.

Referring to FIGS. 4A-4C, the body 422 can include a first or distal connector portion 424a and a second or proximal connector portion 424b opposite (e.g., directly opposite) the first connector portion 424a. The body 422 defines a chamber 426 (e.g., a slot, an opening, a channel) at least partially between the first connector portion 424a and the second connector portion 424b, and a lumen 428 extending the through the body 422, e.g., between and/or fully through the first connector portion 424a and the second connector portion 424b. In at least some embodiments, the lumen 428 has a same dimension as the lumen 108 of the catheter 106 (FIG. 1). The body 422 can further define one or more bores 423, individually identified as a first or left-side bore 423a and a second or right-side bore 423b. Each of the bores 423 can extend in a non-parallel direction relative to the lumen 228, e.g., perpendicular or at least generally perpendicular to the lumen 228.

The gating assembly 440 can include a valve component 442, a first or upper handle 446a positioned on a first side of the valve component 442, a second or lower handle 446b positioned on a second side of the valve component 442 opposite the first side, and one or more handle guides 447 (individually identified as a first or left-side handle guide 447a and a second or right-side handle guide 447b) extending between the first handle 446a and the second handle 446b. Each of the handle guides 447 can be positioned on a side of the valve component 442. For example, in the illustrated embodiment the first handle guide 447a is positioned on a left side of the valve component 442 and the second handle guide 447b is positioned on a right side of the valve component 442, opposite the left side. Each of the handle guides 447 can be slidably received within a corresponding one of the bores 423. For example, in the illustrated embodiment the first handle guide 447a is slidably received within the first bore 423a and the second handle guide 447b is slidably received within the second bore 423b. Accordingly, the handle guides 447 can allow a user to move (e.g., slide) the first and second handles 446a, b relative to the body 422, e.g., upwardly or downwardly, to correspondingly move the gating assembly 440 relative to the body 422.

The valve component 442 of the gating assembly 440 can be positioned within the chamber 426 and configured to move in tandem with the handles 446a-b and handle guides 447. For example, in the illustrated embodiment, the valve component 442 is coupled to the first handle 446a by a shaft 451, such that moving the first handle 446a causes a corresponding movement of the valve component 442. In the closed configuration 421a (FIG. 4B), the valve component 442 is positioned (e.g., lowered) between the first and second connector portions 424a, 424b to inhibit or even prevent (e.g., block) fluid flow through the lumen 428. Similarly, in the open configuration 421b shown in FIG. 4C, the valve component 442 is positioned (e.g., raised) above the first and second connector portions 424a, 424b such that the valve component 442 is not positioned between the first and second connector portions 424a, 424b to allow fluid flow through the lumen 428. In these and/or other embodiments, the valve component 442 can be coupled to the second handle 446b and/or one or more of the handle guides 447. In some embodiments, one or both of the first handle 446a and the second handle 446b include one or more actuation tabs 453 to assist the user in moving the first handle 446a and/or the second handle 446b relative to the body 422. In the illustrated embodiment, for example, the first handle includes two actuation tabs 453, one on a distal side and another on a proximal side, that may be pushed and/or pulled to move the first handle 446a and/or second handle 446b.

In operation, the handle guides 447 and the bores 423 can allow a user to move (e.g., vertically) the gating assembly 440 relative to the body 422 to transition the fluid control device between the open configuration 421b (FIG. 4C) and the closed configuration 421a (FIG. 4B). For example, in the open configuration 421b, the second handle 446b can contact the body 422 and the first handle 446a can be spaced apart from the body 422. A user can press the first handle 446a toward and/or into contact with the body 422 and/or pull the second handle 446b out of contact with and/or away from the body 422 to transition the fluid control device 420 from the open configuration 421b toward and/or to the closed configuration 421a. This is shown by arrows A in FIG. 4C. In some embodiments, the first handle 446a can contact the body 422 in the closed configuration 421a. Additionally, or alternatively, the second handle 446b can contact the body 422 in the open configuration 421b. Accordingly, the body 422 can provide a physical “stop” that indicates to the user when the fluid control device 420 is in the closed configuration 421a, the open configuration 421b, and/or some other intermediate configuration therebetween. Moving the handles 446a-b can reposition the valve component 442 within the chamber 426, e.g., to allow, inhibit, or even prevent fluid flow through the fluid control device 420. This is described in greater detail below with reference to FIGS. 4D and 4E.

Referring to FIG. 4D, the valve component 442 and the shaft 451 can together define a lumen 444. A side port or valve 456 can be coupled to the shaft 451 and configured to control access to the lumen 444. In some embodiments, the side port 456 is a needleless injection port fluidly coupled to the lumen 444. In other embodiments, the side port 456 can include one or more other ports and/or valves. In these and/or other embodiments, fluid can be injected into the lumen 444 via the needleless injection port, as shown by arrow F in FIG. 4D.

The valve component 442 can include a first or distal face 443a and a second or proximal face 443b opposite the first face 443a. The first face 443a can include a first annular retaining structure 436a configured to receive a first sealing element 448a (e.g., O-ring) and/or the second face 443b can include a second annular retaining structure 436b configured to receive a second sealing element 448b (O-ring). When the fluid control device 420 is in the closed configuration 421a as shown in FIG. 4D, the first sealing element 448a can be positioned to contact a first surface 430a within the body 422 at least partially defining the chamber 426 to inhibit or even prevent fluid flow between the first connector portion 424a, the second connector portion 424b, and/or the chamber 426. For example, the lumen 428 can include a first or distal opening 432a formed through the first surface 430a, and the first sealing element 448a can form a first seal 434a around the first opening 432a between the first surface 430a and the first face 443a of the valve component 442. Additionally, or alternatively, the second sealing element 448b can be positioned to contact a second surface 430b within the body 422 at least partially defining the chamber 426 to inhibit or even prevent fluid flow between the first connector portion 424a, the second connector portion 424b, and/or the chamber 426. For example, the lumen 428 can include a second or proximal opening 232b formed through the second surface 430b, and the second sealing element 448b can form a second seal 434b around the second opening 432b between the second surface 430b and the second face 443b of the valve component 442.

Referring to FIGS. 4C and 4D, in some embodiments, the fluid control device 420 includes a third sealing element 448c configured to inhibit, or even prevent fluid from exiting (e.g., leaking from) the chamber 426. For example, the third sealing element 448c can be configured to form a third seal 434c with the shaft 451. The third sealing element 448c can be held at least partially between a first or upper plate 449a and a second or lower plate 449b. In the illustrated embodiment, the second plate 449b defines a recess 436c configured to receive all, or at least a portion, of the third sealing element 448c. In these and/or other embodiments, the first plate 449a can define all, or at least a portion, of the recess 436c. The first plate 449a and the second plate 449b can be coupled to one another and/or to the body 422 to at least partially define a third or upper surface 430c at least partially defining the chamber 426. In at least some embodiments of the present technology, the first seal 434a and/or the second seal 434b are expected to prevent, or at least partially prevent, fluid from entering the chamber 426 at least when the fluid control device 420 is in the closed configuration 421a. The shaft 451 can extend through openings in the first plate 449a and the second plate 449b to contact the third sealing element 448c and form the third seal 434c. In some instances, however, such as when the fluid control device 420 is transitioned from the closed configuration 421a toward the open configuration 421b, fluid within one or both of the first connector portion 424a and the second connector portion 424b may enter the chamber 426. In these and/or other instances, the third seal 434c can be a backup seal that inhibits or even prevents any such fluid that enters the chamber 426 from leaking out of the body 422.

In some embodiments, one or more of the first face 443a, the second face 443b, the first surface 430a, and the second surface 430b are sloped or tapered. In the illustrated embodiment, for example, each of the first face 443a, the second face 443b, the first surface 430a, and the second surface 430b are sloped relative to a vertical axis V of the fluid control device 420. As shown in FIG. 4E, the first face 443a is at a first angle A1 relative to the vertical axis V, the second face 443b is at a second angle A2 relative to the vertical axis V, the first surface 430a is at a third angle A3 relative to the vertical axis V, and the second surface 430b is at a fourth angle A4 relative to the vertical axis V. Individual ones of the first angle A1, the second angle A2, the third angle A3, and/or the fourth angle A4 can be the same or different than one or more others of the first angle A1, the second angle A2, the third angle A3, and/or the fourth angle A4. In the illustrated embodiment, for example, the first angle A1, the second angle A2, the third angle A3, and the fourth angle A4 are the same. In other embodiments, any one, two, or three of the first angle A1, the second angle A2, the third angle A3, and the fourth angle A4 can be different than one or more of the first angle A1, the second angle A2, the third angle A3, and/or the fourth angle A4. In these and/or other embodiments, each of the first angle A1, the second angle A2, the third angle A3, and the fourth angle A4 can be up to and/or about equal to 0.5 degrees, 0.75 degrees, 1 degree, 2 degrees, 3 degrees, 4 degrees, 5 degrees, 10 degrees, 15 degrees, 20 degrees, 30 degrees, 40 degrees, 50 degrees, 60 degrees, 70 degrees, or another suitable angle between any two or more of the above-listed angles. In some embodiments, the first through fourth angles A1-A4 can be about 0.75 degrees. In these and/or other embodiments, one or more of the first face 443a, the second face 443b, the first surface 430a, and the second surface 430b can be sloped relative to another axis and/or parallel to, or at least generally parallel to, the vertical axis V and/or another axis.

In some aspects of the present technology, the slope of the first face 443a, the second face 443b, the first surface 430a, and/or the second surface 430b is expected to improve the quality of the seals 434a, 434b formed between the first face 443a and the first surface 430a and the second face 443b and the second surface 430b, respectively. For example, the slope of the first face 443a, the second face 443b, the first surface 430a, and/or the second surface 430b is expected to press (e.g., compress) the first sealing element 448a and/or the second sealing element 448b between the valve component 442 and the corresponding surfaces 430a-b within the body 422 as the valve component 442 is lowered between the connector portions 424a, 424b to transition the fluid control device 420 to the closed configuration 421a. Additionally, or alternatively, the slope of the first face 443a, the second face 443b, the first surface 430a, and/or the second surface 430b is expected to reduce resistance to transitioning the fluid control device 420 between the closed configuration 421a and the open configuration 421b. In these and/or other embodiments, the slope of the first face 443a, the second face 443b, the first surface 430a, and/or the second surface 430b is expected to reduce or eliminate compression on the sealing elements 448a-b when the fluid control device 420 is in the open configuration 421b. More specifically, the sealing elements 448a-b do not contact or significantly contact the first and second surfaces 430a-b when the valve component 442 is raised to transition the fluid control device 420 to the open configuration 421b.

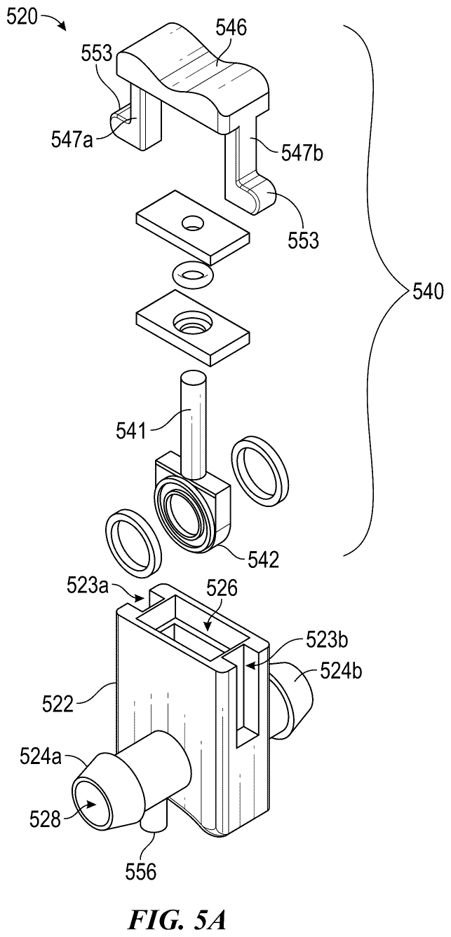

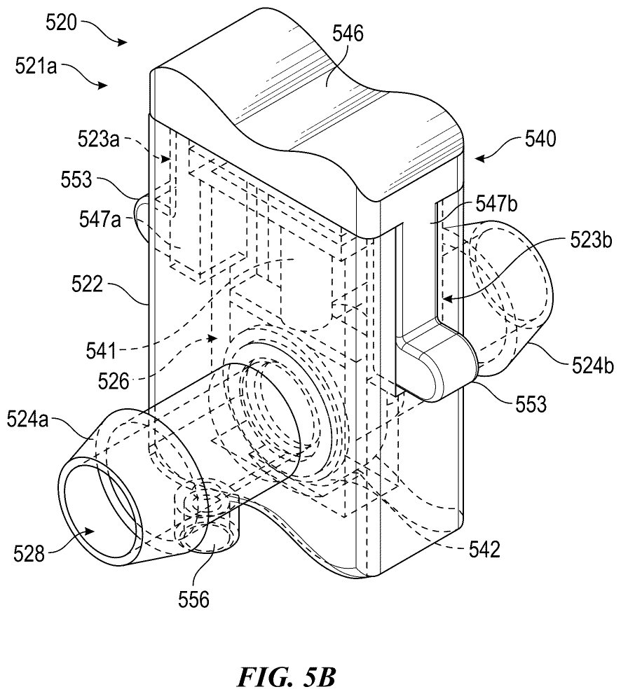

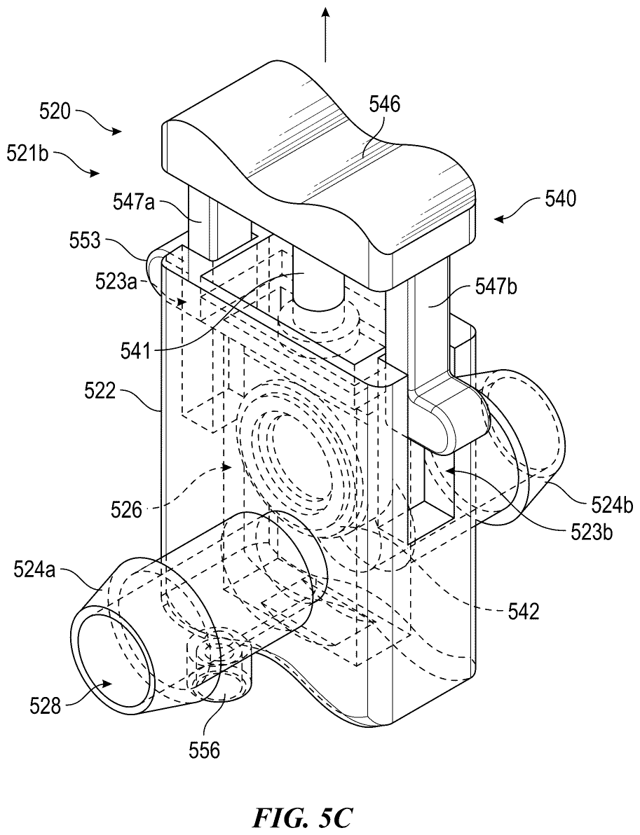

FIGS. 5A-5C are different views of a fluid control device 520 configured in accordance with additional embodiments of the present technology. FIG. 5A is an exploded view of the fluid control device 520. FIG. 5B is a perspective view of the fluid control device 520 in a closed configuration 521a. FIG. 5C is a perspective view of the fluid control device 520 in an open configuration 521b. Referring to FIGS. 5A-5C, the fluid control device 520 can include at least some features that are at least generally similar or identical in structure and/or function to the fluid control device 120 of FIG. 1, the fluid control device 220 of FIGS. 2A-3, and/or the fluid control device 420 of FIGS. 4A-4E. For example, in the illustrated embodiment the fluid control device 520 includes a body 522 and a gating assembly 540. The body 522 includes a first connector portion 524a and a second connector portion 524b, and defines a chamber 526 within the body 522 and a lumen 528 extending through the first connector portion 524a and the second connector portion 524b. In at least some embodiments, the lumen 528 has a same dimension as the lumen 108 of the catheter 106 (FIG. 1). The gating assembly 540 includes a valve component 542 coupled to a handle 546 via a shaft 541. The valve component 542 can include at least some or all of the features and/or functionality of the valve component 442 described in detail above with reference to FIGS. 4A-4E. However, in the illustrated embodiment the valve component 542 and the shaft 541 of the fluid control device 520 do not define a lumen fluidly connected to a port or valve. Instead, the first connector portion 524a includes a side port 556 that can be at least generally similar to the side port 256 described in detail above with reference to at least FIG. 3. Additionally, the fluid control device 520 can have a smaller form factor than the fluid control device 420 of FIGS. 4A-4E, which is expected to make the fluid control device 520 easier to handle and/or operate.

The handle 546 further includes one or more handle guides 547 (individually identified as a first or left-side handle guide 547a and a second or right-side handle guide 547b) that extend downwardly from the handle 546 toward one or both of the connector portions 524a, 524b. The handle 546 and/or individual ones of the handle guides 547 can include at least some or all of the features and/or functionality of the handle 446 and/or the handle guides 447 described in detail above with reference to FIGS. 4A-4E. However, the first handle guide 547a and the second handle guide 547b can each be received within a corresponding slot or channel 523a, 523b defined by the body 522. In some embodiments, one or more of the handle guides 547 include one or more actuation tabs 553, individual ones of which can be coupled to one of the handle guides 547, e.g., at or near an end opposite the handle 546. Accordingly, in at least some embodiments, each of the handle guides 547 can be coupled to the handle 546 at or near a first end and include the tab 553 at or near a second end opposite the first end. During a procedure, a user can move (e.g., raise or lower) the handle 546 relative to the body 522 (e.g., by pushing and/or pulling the actuation tabs 553) to transition the fluid control device 520 between the closed configuration 521a and the open configuration 521b.

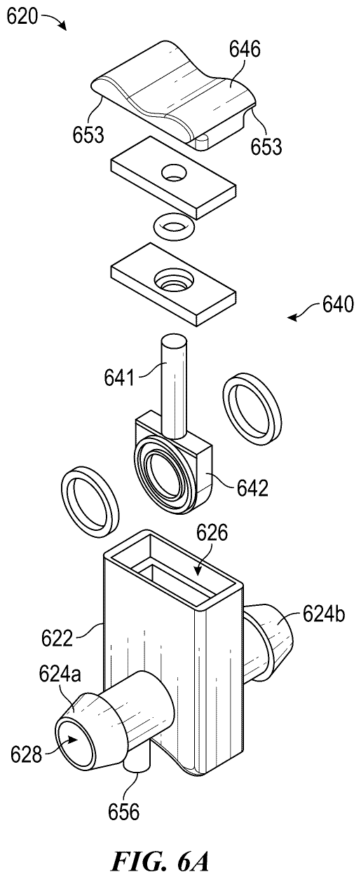

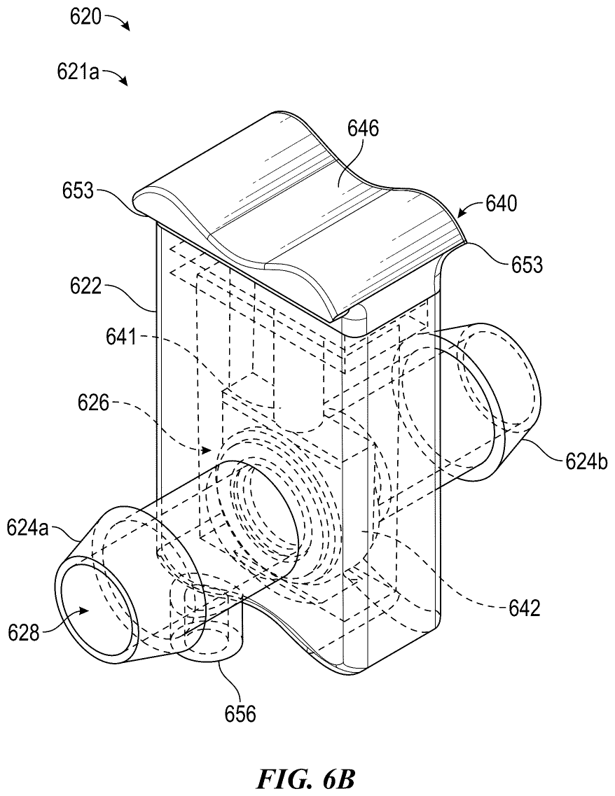

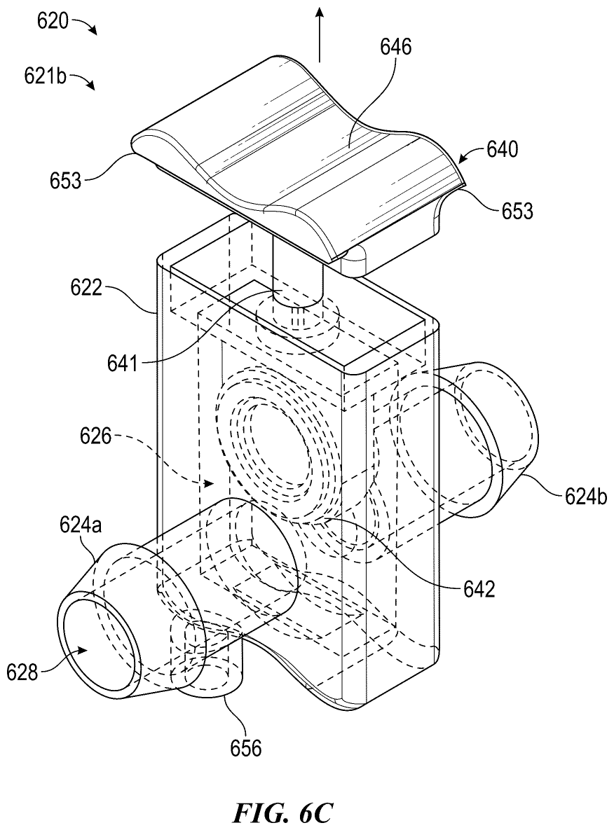

FIGS. 6A-6C are different views of a fluid control device 620 configured in accordance with additional embodiments of the present technology. FIG. 6A is an exploded view of the fluid control device 620. FIG. 6B is a perspective view of the fluid control device 620 in a closed configuration 621a. FIG. 6C is a perspective view of the fluid control device 620 in an open configuration 621b. Referring to FIGS. 6A-6C, the fluid control device 620 can include at least some features that are at least generally similar or identical in structure and/or function to the fluid control device 120 of FIG. 1, the fluid control device 220 of FIGS. 2A-3, the fluid control device 420 of FIGS. 4A-4E, and/or the fluid control device 520 of FIGS. 5A-5C. For example, the fluid control device 620 includes a body 622 and a gating assembly 640. The body 622 includes a first connector portion 624a, a second connector portion 624b, and defines a chamber 626 within the body 622 and a lumen 628 extending through the first connector portion 624a and the second connector portion 624b. In at least some embodiments, the lumen 628 has a same dimension as the lumen 108 of the catheter 106 (FIG. 1). The gating assembly 640 includes a valve component 642 coupled to a handle 646 via a shaft 641. The valve component 642 can include at least some or all of the features and/or functionality of the valve component 542 described in detail above with reference FIGS. 5A-5C. For example, the valve component 642 and the shaft 641 of the fluid control device 620 do not define a lumen fluidly connected to a port or valve, and the first connector portion 624a includes a side port 656 configured to be coupled to a source of fluid. Additionally, the fluid control device 620 can have a smaller form factor than the fluid control device 420 of FIGS. 4A-4E and/or the fluid control device 520 of FIGS. 5A-5C, which is expected to make the fluid control device 620 easier to handle and/or operate.

The handle 646 can include at least some or all of the features and/or functionality of the handle 546 described in detail above with reference to FIGS. 5A-5C. However, the handle 646 does not include handle guides and instead includes integrated actuation tabs 653 at least generally similar to the actuation tabs 453 of the fluid control device 420 described in detail above with reference to FIGS. 4A-4E. The handle 646 and/or the actuation tabs 653 can be positioned on a same side of the body 622 and/or the chamber 626 in both the closed configuration 621a and the open configuration 621b. During a procedure, a user can move (e.g., raise or lower) the handle 646 relative to the body 622 (e.g., by pushing on or pulling the actuation tabs 653) to transition the fluid control device between the closed configuration 621a and the open configuration 621b.

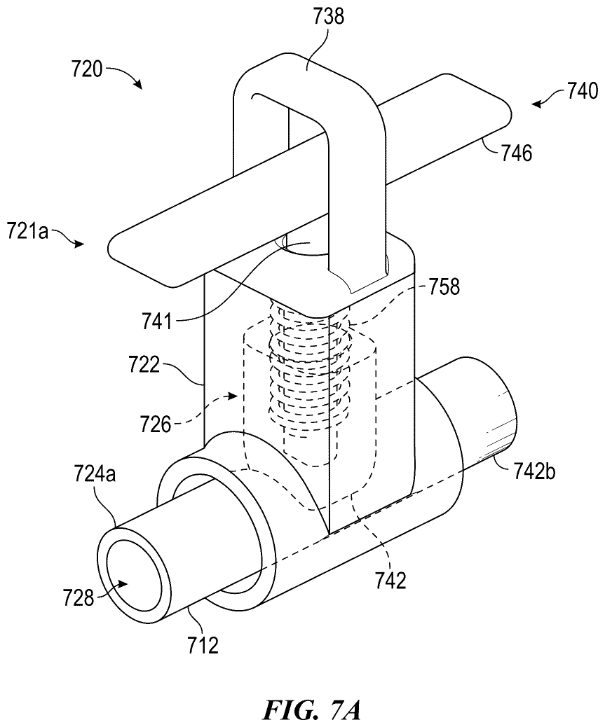

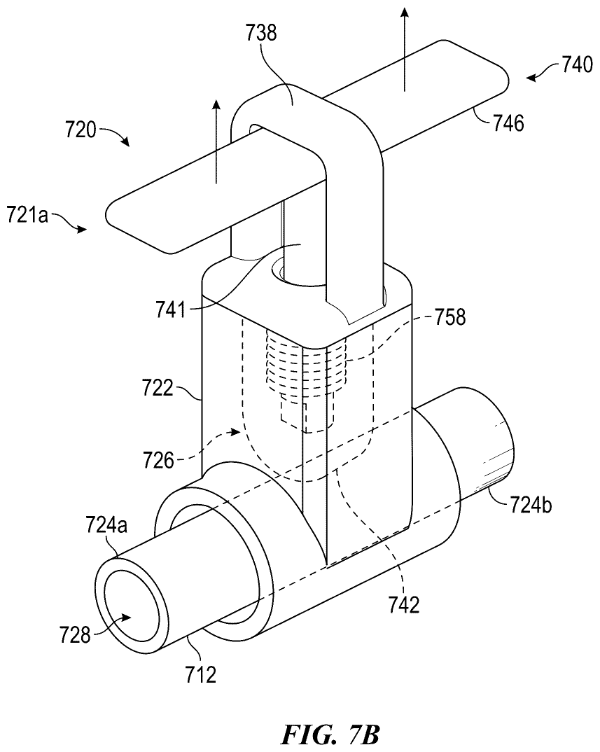

FIGS. 7A and 7B are different views of another fluid control device 720 configured in accordance with embodiments of the present technology. FIG. 7A is a perspective view of the fluid control device 720 in a closed configuration 721a. FIG. 7B is a perspective view of the fluid control device 720 in an open configuration 721b. Referring to FIGS. 7A and 7B together, the fluid control device 720 can include at least some features that are at least generally similar or identical in structure and/or function to the fluid control device 120 of FIG. 1, the fluid control device 220 of FIGS. 2A-3, the fluid control device 420 of FIGS. 4A-4E, the fluid control device 520 of FIGS. 5A-5C, and/or the fluid control device 620 of FIGS. 6A-6C. For example, the fluid control device 720 includes a body 722 and a gating assembly 740. The body 722 can include a first connector portion 724a and a second connector portion 724b and define a chamber 726 within the body 722. A lumen 728 extends between the first connector portion 724a and the second connector portion 724b.

In the illustrated embodiment, the first connector portion 724a and/or the second connector portion 724b are part of a tubing section 712 received through the body 722 (e.g., positioned at least partially within and/or extending through the chamber 726) that can be connected to one or more other tubing sections 112 in the tubing assembly 110 (FIG. 1). In other embodiments, the tubing section 712 and connector portions 724a-b can be omitted and one of the tubing sections 112a, 112b (FIG. 1) of the tubing assembly 110 (FIG. 1) can be received through the body 722 and/or define the lumen 728. The fluid control device 720 can have a relatively small form factor which is expected to make the fluid control device 720 easier to handle and/or operate.

The gating assembly 740 includes a valve component 742 coupled to a handle 746 via a shaft 741. The valve component 742 can include at least some or all of the features and/or functionality of the valve component 642 described in detail above with reference FIGS. 6A-6C. For example, the valve component 742 and the shaft 741 of the fluid control device 720 do not define a lumen fluidly connected to a port or valve. Additionally, the gating assembly 740 can include a biasing element 758 (e.g., a spring) operably coupled to the valve component 742. In the illustrated embodiment, for example, the biasing element 758 is positioned around the shaft 741 and configured to bias the fluid control device 720 toward and/or into the closed configuration 721a (FIG. 7A). In other embodiments, the biasing element 758 can be configured to bias the fluid control device 720 toward and/or into the open configuration 721b (FIG. 7B). In the closed configuration 721a (FIG. 7A), the valve component 742 can press against the tubing section 712 (or other tubing section positioned through the body 722) to deform (e.g., collapse) a portion of the tubing section 712 to thereby seal the lumen 728, inhibiting or even preventing fluid flow through the lumen 728. In the open configuration 721b (FIG. 7B), the valve component 742 can be moved (e.g., upwardly) away from the tubing section to allow the tubing section 712 to assume its manufactured or undeformed shape, e.g., to thereby allow fluid flow through the lumen 728. In some embodiments, the tubing section 712 can undergo elastic or at least generally elastic deformation and can automatically open to allow fluid flow when the fluid control device 720 is transitioned toward and/or into the open configuration 721b (FIG. 7B). In these and/or other embodiments, the valve component 742 can be coupled to the tubing section 712 so that moving the valve component 742 applies a force to the tubing section 712 that opens the tubing section 712, e.g., to allow fluid flow through the lumen 728.

In operation, a user can move (e.g., raise or lower) the handle 746 relative to the body 722 to transition the fluid control device 720 between the closed configuration 721a and the open configuration 721b. For example, the user can move the handle 746 away from the body 722 to transition the fluid control device 720 toward and/or to the open configuration 721b and/or move the handle 746 toward the body 722 to transition the fluid control device 720 toward and/or to the closed configuration 721a. For embodiments in which the fluid control device 720 is biased toward one of the closed configuration 721a or the open configuration 721b, the biasing element 758 can return the fluid control device 720 to that configuration without the user moving the handle 746. For example, the biasing element 758 biases the handle 746 and the valve component 742 downwardly in FIGS. 7A and 7B to compress/pinch the tubing section 712 to seal the lumen 728. For embodiments in which the valve component 742 is coupled to the tubing section, moving the handle 746 can open the tubing section 712 (e.g., with the movement of the handle 746 directly opening the tubing section 712) to allow fluid to flow through the lumen 728. In other embodiments, moving the valve component 742 can allow the tubing section 712) to open (e.g., without the movement of the handle 746 directly opening the tubing section 712) and thereby allow fluid to flow through the lumen 728.

Several aspects of the present technology are set forth in the following examples:

1. A fluid control device for a clot material removal system, the fluid control device comprising:
a body, including—
   a first connector portion,
   a second connector portion opposite the first connector portion, and
   an inner surface at least partially defining a chamber, the body further defining a lumen extending through the first connector portion and the second connector portion; and
a gating assembly, including—
   a handle positioned outside the chamber and movable between a first position and second position, and
   a valve component coupled to the handle and positioned within the chamber,
wherein—
   with the handle in the first position, the valve component is positioned between the first connector portion and the second connector portion to block fluid flow through the lumen between the first connector portion and the second connector portion, and
   with the handle in the second position, the valve component is positioned to allow fluid flow through the lumen between the first connector portion and the second connector portion.

2. The fluid control device of example 1 wherein the handle is a first handle positioned on a first side of the body, the gating assembly further including a second handle positioned outside the chamber on a second side of the body opposite the first side, wherein
with the first handle in the first position, the first handle contacts the body and the second handle is spaced apart from the body, and
with the first handle in the second position, the first handle is spaced apart from the body and the second handle contacts the body.

3. The fluid control device of example 1 wherein—
the gating assembly further includes a handle guide coupled to the handle, and
the body further defines a bore configured to slidably receive the handle guide to facilitate transitioning the handle between the first position and the second position.

4. The fluid control device of example 1 wherein—
the gating assembly further includes a handle guide coupled to the handle, and
the body further defines a slot through an exterior surface of the body configured to slidably receive the handle guide to facilitate transitioning the handle between the first position and the second position.

5. The fluid control device of example 4 wherein a first end of the handle guide is coupled to the handle, and wherein a second end of the handle guide opposite the first end includes an actuation tab extending outwardly away from the slot.

6. The fluid control device of example 1 wherein the body further defines an opening, and wherein gating assembly further includes a shaft extending through the opening between the handle and the valve component.

7. The fluid control device of example 6 wherein—
the valve component and the shaft together define a gating assembly lumen in fluid communication with the chamber and/or the lumen of the body, and
the gating assembly further includes an injection port fluidly coupled to the gating assembly lumen.

8. The fluid control device of example 1 wherein one of the first connector portion and the second connector portion include an injection port.

9. The fluid control device of example 1 wherein the valve component includes an angled sealing surface, and wherein, with the handle in the first position, the angled sealing surface contacts the inner surface of the body to form an at least substantially fluid impermeable seal therewith to block the fluid flow.

10. The fluid control device of example 9 wherein, relative to a vertical axis of the fluid control device, the angled sealing surface is at an angle of up to 15 degrees.

11. The fluid control device of example 10 wherein the angle is 0.75 degrees.

12. The fluid control device of example 9 wherein, relative to a vertical axis of the fluid control device, the inner surface of the body is at a first angle and the angled sealing surface is at a second angle.

13. The fluid control device of example 12 wherein the first angle is equal to the second angle.

14. The fluid control device of example 9 wherein the angled sealing surface includes a sealing element coupled to a face of the valve component.

15. The fluid control device of example 9 wherein the angled sealing surface is a first angled sealing surface on a first side of the valve component, and wherein the valve component further includes a second angled sealing surface on a second side of the valve component opposite the first side.

16. A system for removing clot material from within a patient, the system comprising:
the fluid control device of any of examples 1-15;
a catheter coupled to one of the first connector portion and the second connector portion; and a pressure source coupled to the other of the first connector portion and the second connector portion and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

17. The system of example 16 wherein the fluid control device and the catheter define a fluid path to the pressure source having an at least generally uniform inner diameter.

18. A fluid control device for a clot material removal system, the fluid control device comprising:
a body, including—
   a first connector portion,
   a second connector portion opposite the first connector portion,
   one or more inner surfaces at least partially defining a chamber, the body further defining a lumen extending through the first connector portion and the second connector portion,
   a first sealing element positioned within the chamber, and
   a second sealing element positioned within the chamber; and
a gating component, including—
   a handle positioned outside the chamber and movable between a first position and a second position, and
   a valve component coupled to the handle, positioned within the chamber in contact with the first sealing element and the second sealing element, and spaced apart from at least one of the one or more inner surfaces, wherein the valve component defines a valve component lumen,
wherein—
   with the handle in the first position, the valve component lumen is rotated out of alignment with the lumen to inhibit or prevent fluid flow through the lumen between the first connector portion and the second connector portion, and
   with the handle in the second position, the valve component lumen is aligned with the lumen to allow fluid flow through the lumen between the first connector portion and the second connector portion.

19. The fluid control device of example 18 wherein—
the one or more inner surfaces include a first surface defining a first opening to the lumen through the first connector portion and a second surface defining a second opening to the lumen through the second connector portion, and
the valve component is spaced apart from the first surface and the second surface.

20. The fluid control device of example 19 wherein—
the first sealing element is coupled to the first surface,
the second sealing element is coupled to the second surface, and
the valve component contacts the first sealing element and the second sealing element to form substantially fluid-impermeable seals therewith.

21. The fluid control device of example 20 wherein one of or both the first sealing element and the second sealing element are configured to space the valve component apart from the one or more inner surfaces.

22. The fluid control device of example 18 wherein the chamber is cylindrical with a rectangular cross-sectional shape and the valve component has a spherical shape.

23. A system for removing clot material from within a patient, the system comprising:
the fluid control device of any of examples 18-22;
a catheter coupled to one of the first connector portion and the second connector portion; and
a pressure source coupled to the other of the first connector portion and the second connector portion and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

24. The system of example 23 wherein the fluid control device and the catheter define a fluid path to the pressure source having an at least generally uniform inner diameter.

25. A fluid control device for a clot material removal system, the fluid control device comprising:
a body defining a chamber and configured to receive a tubing section at least partially within and/or through the chamber; and
a gating assembly, including—
   a handle positioned outside the chamber and movable between a first position and second position, and
   a valve component coupled to the handle and positioned within the chamber,
wherein—
   with the handle in the first position, the valve component is configured to close the tubing section to at least partially prevent fluid flow therethrough, and
   with the handle in the second position, the valve component is configured to allow fluid flow through the tubing section.

26. The fluid control device of example 25 wherein the handle is closer to the body in the first position than in the second position.

27. The fluid control device of example 25 wherein the body further defines an opening, and wherein gating assembly further includes a shaft extending through the opening between the handle and the valve component.

28. The fluid control device of example 25 wherein the valve component is configured to be coupled to the tubing section so that moving the handle to the second position opens the tubing section to fluid flow.

29. The fluid control device of example 25 wherein, with the handle in the second position, the valve component is positioned to allow the tubing section to open.

30. The fluid control device of example 25 wherein, with the handle in the first position, the valve component is configured to press against and/or cause an elastic deformation of the tubing section.

31. A system for removing clot material from within a patient, the system comprising:
the fluid control device of any of examples 25-30;
a catheter fluidly coupled to the tubing section on a first side of the fluid control device; and
a pressure source fluidly coupled to the tubing section on a second side of the fluid control device, opposite the catheter, and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

32. The system of example 31 wherein the fluid control device and the catheter define a fluid path to the pressure source having an at least generally uniform inner diameter.

33. The system of example 31 wherein the tubing section is a first tubing section having a first end portion and a second end portion, and wherein the system further comprises:
a second tubing section configured to connect the first end portion to the catheter, and
a third tubing section configured to connect the second end portion to the pressure source.

34. A system for removing clot material from within a patient, the system comprising:
a fluid control device, including—
   a body having—
      a first connector portion,
      a second connector portion opposite the first connector portion, and
      an inner surface at least partially defining a chamber, the body further defining a lumen extending through the first connector portion and the second connector portion; and
   a gating assembly having—
      a handle positioned outside the chamber and movable between a first position and second position, and
      a valve component coupled to the handle and positioned within the chamber,
   wherein—
      with the handle in the first position, the valve component is positioned between the first connector portion and the second connector portion to block fluid flow through the lumen between the first connector portion and the second connector portion, and
      with the handle in the second position, the valve component is positioned to allow fluid flow through the lumen between the first connector portion and the second connector portion.

35. The system of example 34 wherein the handle is a first handle positioned on a first side of the body, the gating assembly further including a second handle positioned outside the chamber on a second side of the body opposite the first side, wherein—
with the first handle in the first position, the first handle contacts the body and the second handle is spaced apart from the body, and
with the first handle in the second position, the first handle is spaced apart from the body and the second handle contacts the body.

36. The system of example 34 or 35 wherein—
the gating assembly further includes a handle guide coupled to the handle, and
the body further defines a bore configured to slidably receive the handle guide to facilitate transitioning the handle between the first position and the second position.

37. The system of any of examples 34-36 wherein—
the gating assembly further includes a handle guide coupled to the handle, and
the body further defines a slot through an exterior surface of the body configured to slidably receive the handle guide to facilitate transitioning the handle between the first position and the second position.

38. The system of example 37 wherein a first end of the handle guide is coupled to the handle, and wherein a second end of the handle guide opposite the first end includes an actuation tab extending outwardly away from the slot.

39. The system of any of examples 34-38 wherein the body further defines an opening, and wherein gating assembly further includes a shaft extending through the opening between the handle and the valve component.

40. The system of example 39 wherein—
the valve component and the shaft together define a gating assembly lumen in fluid communication with the chamber and/or the lumen of the body, and
the gating assembly further includes an injection port fluidly coupled to the gating assembly lumen.

41. The system of any of examples 34-40 wherein one of the first connector portion and the second connector portion include an injection port.

42. The system of any of examples 34-41 wherein the valve component includes an angled sealing surface, and wherein, with the handle in the first position, the angled sealing surface contacts the inner surface of the body to form an at least substantially fluid impermeable seal therewith to block the fluid flow.

43. The system of example 42 wherein, relative to a vertical axis of the fluid control device, the angled sealing surface is at an angle of up to 15 degrees.

44. The system of example 43 wherein the angle is 0.75 degrees.

45. The system of any of examples 42-44 wherein, relative to a vertical axis of the fluid control device, the inner surface of the body is at a first angle and the angled sealing surface is at a second angle.

46. The system of example 45 wherein the first angle is equal to the second angle.

47. The system of any of examples 42-46 wherein the angled sealing surface includes a sealing element coupled to a face of the valve component.

48. The system of any of examples 42-47 wherein the angled sealing surface is a first angled sealing surface on a first side of the valve component, and wherein the valve component further includes a second angled sealing surface on a second side of the valve component opposite the first side.

49. The system of any of examples 34-48, further comprising:
a catheter coupled to one of the first connector portion and the second connector portion; and
a pressure source coupled to the other of the first connector portion and the second connector portion and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

50. The system of example 49 wherein the fluid control device and the catheter define a fluid path to the pressure source having an at least generally uniform inner diameter.

51. A system for removing clot material from within a patient, the system comprising:
a fluid control device, including—
   a body having—
      a first connector portion,
      a second connector portion opposite the first connector portion,
      one or more inner surfaces at least partially defining a chamber, the body further defining a lumen extending through the first connector portion and the second connector portion,
      a first sealing element positioned within the chamber, and
      a second sealing element positioned within the chamber; and
   a gating component having—
      a handle positioned outside the chamber and movable between a first position and a second position, and
      a valve component coupled to the handle, positioned within the chamber in contact with the first sealing element and the second sealing element, and spaced apart from at least one of the one or more inner surfaces, wherein the valve component defines a valve component lumen,
   wherein—
      with the handle in the first position, the valve component lumen is rotated out of alignment with the lumen to inhibit or prevent fluid flow through the lumen between the first connector portion and the second connector portion, and
      with the handle in the second position, the valve component lumen is aligned with the lumen to allow fluid flow through the lumen between the first connector portion and the second connector portion.

52. The system of example 51 wherein—
the one or more inner surfaces include a first surface defining a first opening to the lumen through the first connector portion and a second surface defining a second opening to the lumen through the second connector portion, and
the valve component is spaced apart from the first surface and the second surface.

53. The system of example 52 wherein—
the first sealing element is coupled to the first surface,
the second sealing element is coupled to the second surface, and
the valve component contacts the first sealing element and the second sealing element to form substantially fluid-impermeable seals therewith.

54. The system of example 53 wherein one of or both the first sealing element and the second sealing element are configured to space the valve component apart from the one or more inner surfaces.

55. The system of any of examples 51-54 wherein the chamber is cylindrical with a rectangular cross-sectional shape and the valve component has a spherical shape.

56. The system of any of examples 51-55, further comprising:
a catheter coupled to one of the first connector portion and the second connector portion; and
a pressure source coupled to the other of the first connector portion and the second connector portion and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

57. The system of example 56 wherein the fluid control device and the catheter define a fluid path to the pressure source having an at least generally uniform inner diameter.

58. A system for removing clot material from within a patient, the system comprising:
a fluid control device, including—
   a body defining a chamber and configured to receive a tubing section at least partially within and/or through the chamber; and
   a gating assembly, including—
      a handle positioned outside the chamber and movable between a first position and second position, and
      a valve component coupled to the handle and positioned within the chamber,
   wherein—
      with the handle in the first position, the valve component is configured to close the tubing section to at least partially prevent fluid flow therethrough, and
      with the handle in the second position, the valve component is configured to allow fluid flow through the tubing section.

59. The system of example 58 wherein the handle is closer to the body in the first position than in the second position.

60. The system of example 58 or example 59 wherein the body further defines an opening, and wherein gating assembly further includes a shaft extending through the opening between the handle and the valve component.

61. The system of any of examples 58-60 wherein the valve component is configured to be coupled to the tubing section so that moving the handle to the second position opens the tubing section to fluid flow.

62. The system of any of examples 58-61 wherein, with the handle in the second position, the valve component is positioned to allow the tubing section to open.

63. The system of any of examples 58-62 wherein, with the handle in the first position, the valve component is configured to press against and/or cause an elastic deformation of the tubing section.

64. The system of any of examples 58-63, further comprising:
a catheter fluidly coupled to the tubing section on a first side of the fluid control device; and
a pressure source fluidly coupled to the tubing section on a second side of the fluid control device, opposite the catheter, and configured to generate and store a vacuum,
wherein—
   with the handle in the first position, the fluid control device prevents the vacuum from being applied to the catheter, and
   with the handle in the second position, the fluid control device allows the vacuum to be applied to the catheter to remove clot material from the patient.

65. The system of example 64 wherein the fluid control device and the catheter define a fluid path to the pressure source having an at least generally uniform inner diameter.

66. The system of example 64 or example 65 wherein the tubing section is a first tubing section having a first end portion and a second end portion, and wherein the system further comprises:
a second tubing section configured to connect the first end portion to the catheter, and
a third tubing section configured to connect the second end portion to the pressure source.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for removing clot material from within a patient, the system comprising:
a fluid control device, including—
a body having—
a first connector portion,
a second connector portion opposite the first connector portion, and
an inner surface at least partially defining a chamber, the body further defining a lumen extending through the first connector portion and the second connector portion; and
a gating assembly having—
a handle positioned outside the chamber and movable between a first position and second position, and
a valve component coupled to the handle and positioned within the chamber,
wherein—
with the handle in the first position, the valve component is positioned between the first connector portion and the second connector portion to block fluid flow through the lumen between the first connector portion and the second connector portion, and
with the handle in the second position, the valve component is positioned to allow fluid flow through the lumen between the first connector portion and the second connector portion.

2. The system of claim 1 wherein the handle is a first handle positioned on a first side of the body, the gating assembly further including a second handle positioned outside the chamber on a second side of the body opposite the first side, wherein
with the first handle in the first position, the first handle contacts the body and the second handle is spaced apart from the body, and
with the first handle in the second position, the first handle is spaced apart from the body and the second handle contacts the body.

3. The system of claim 1 wherein
the gating assembly further includes a handle guide coupled to the handle, and
the body further defines a bore configured to slidably receive the handle guide to facilitate transitioning the handle between the first position and the second position.

4. The system of claim 1 wherein
the gating assembly further includes a handle guide coupled to the handle, and
the body further defines a slot through an exterior surface of the body configured to slidably receive the handle guide to facilitate transitioning the handle between the first position and the second position.

5. The system of claim 4 wherein a first end of the handle guide is coupled to the handle, and wherein a second end of the handle guide opposite the first end includes an actuation tab extending outwardly away from the slot.

6. The system of claim 1 wherein the body further defines an opening, and wherein gating assembly further includes a shaft extending through the opening between the handle and the valve component.

7. The system of claim 6 wherein
the valve component and the shaft together define a gating assembly lumen in fluid communication with the chamber and/or the lumen of the body, and
the gating assembly further includes an injection port fluidly coupled to the gating assembly lumen.

8. The system of claim 1 wherein one of the first connector portion and the second connector portion include an injection port.

9. The system of claim 1 wherein the valve component includes an angled sealing surface, and wherein, with the handle in the first position, the angled sealing surface contacts the inner surface of the body to form an at least substantially fluid impermeable seal therewith to block the fluid flow.

10. The system of claim 9 wherein, relative to a vertical axis of the fluid control device, the angled sealing surface is at an angle of up to 15 degrees.

11. The system of claim 10 wherein the angle is 0.75 degrees.

12. The system of claim 9 wherein, relative to a vertical axis of the fluid control device, the inner surface of the body is at a first angle and the angled sealing surface is at a second angle.

13. The system of claim 12 wherein the first angle is equal to the second angle.

14. The system of claim 9 wherein the angled sealing surface includes a sealing element coupled to a face of the valve component.

15. The system of claim 9 wherein the angled sealing surface is a first angled sealing surface on a first side of the valve component, and wherein the valve component further includes a second angled sealing surface on a second side of the valve component opposite the first side.

16. The system of claim 1, further comprising:
a catheter coupled to one of the first connector portion and the second connector portion; and
a pressure source coupled to the other of the first connector portion and the second connector portion and configured to generate and store a vacuum,
wherein—
with the handle in the first position, the fluid control device prevents the vacuum from being applied to the catheter, and
with the handle in the second position, the fluid control device allows the vacuum to be applied to the catheter to remove clot material from the patient.

17. The system of claim 16 wherein the fluid control device and the catheter define a fluid path to the pressure source having an at least generally uniform inner diameter.

18. A system for removing clot material from within a patient, the system comprising:
a fluid control device, including—
a body having—
a first connector portion,
a second connector portion opposite the first connector portion,
one or more inner surfaces at least partially defining a chamber, the body further defining a lumen extending through the first connector portion and the second connector portion,
a first sealing element positioned within the chamber, and
a second sealing element positioned within the chamber; and
a gating component having—
a handle positioned outside the chamber and movable between a first position and a second position, and
a valve component coupled to the handle, positioned within the chamber in contact with the first sealing element and the second sealing element, and spaced apart from at least one of the one or more inner surfaces, wherein the valve component defines a valve component lumen,
wherein—
with the handle in the first position, the valve component lumen is rotated out of alignment with the lumen to inhibit or prevent fluid flow through the lumen between the first connector portion and the second connector portion, and
with the handle in the second position, the valve component lumen is aligned with the lumen to allow fluid flow through the lumen between the first connector portion and the second connector portion.

19. The system of claim 18 wherein—
the one or more inner surfaces include a first surface defining a first opening to the lumen through the first connector portion and a second surface defining a second opening to the lumen through the second connector portion, and
the valve component is spaced apart from the first surface and the second surface.

20. The system of claim 19 wherein—
the first sealing element is coupled to the first surface,
the second sealing element is coupled to the second surface, and
the valve component contacts the first sealing element and the second sealing element to form substantially fluid-impermeable seals therewith.

21. The system of claim 20 wherein one of or both the first sealing element and the second sealing element are configured to space the valve component apart from the one or more inner surfaces.

22. The system of claim 18 wherein the chamber is cylindrical with a rectangular cross-sectional shape and the valve component has a spherical shape.

23. The system of claim 18, further comprising:
a catheter coupled to one of the first connector portion and the second connector portion; and
a pressure source coupled to the other of the first connector portion and the second connector portion and configured to generate and store a vacuum,
wherein—
with the handle in the first position, the fluid control device prevents the vacuum from being applied to the catheter, and
with the handle in the second position, the fluid control device allows the vacuum to be applied to the catheter to remove clot material from the patient.

24. The system of claim 23 wherein the fluid control device and the catheter define a fluid path to the pressure source having an at least generally uniform inner diameter.

25. A system for removing clot material from within a patient, the system comprising:
a fluid control device, including—
a body defining a chamber and configured to receive a tubing section at least partially within and/or through the chamber; and
a gating assembly, including—
a handle positioned outside the chamber and movable between a first position and second position, and
a valve component coupled to the handle and positioned within the chamber,
wherein—
with the handle in the first position, the valve component is configured to close the tubing section to at least partially prevent fluid flow therethrough, and
with the handle in the second position, the valve component is configured to allow fluid flow through the tubing section.

26. The system of claim 25 wherein the handle is closer to the body in the first position than in the second position.

27. The system of claim 25 wherein the body further defines an opening, and wherein gating assembly further includes a shaft extending through the opening between the handle and the valve component.

28. The system of claim 25 wherein the valve component is configured to be coupled to the tubing section so that moving the handle to the second position opens the tubing section to fluid flow.

29. The system of claim 25 wherein, with the handle in the second position, the valve component is positioned to allow the tubing section to open.

30. The system of claim 25 wherein, with the handle in the first position, the valve component is configured to press against and/or cause an elastic deformation of the tubing section.

31. The system of claim 25, further comprising:
a catheter fluidly coupled to the tubing section on a first side of the fluid control device; and
a pressure source fluidly coupled to the tubing section on a second side of the fluid control device, opposite the catheter, and configured to generate and store a vacuum,
wherein—
with the handle in the first position, the fluid control device prevents the vacuum from being applied to the catheter, and
with the handle in the second position, the fluid control device allows the vacuum to be applied to the catheter to remove clot material from the patient.

32. The system of claim 31 wherein the fluid control device and the catheter define a fluid path to the pressure source having an at least generally uniform inner diameter.

33. The system of claim 31 wherein the tubing section is a first tubing section having a first end portion and a second end portion, and wherein the system further comprises:
a second tubing section configured to connect the first end portion to the catheter, and
a third tubing section configured to connect the second end portion to the pressure source.
